(19) United States
(12) Patent Application Publication
(10) Pub. No.: US 2015/0051480 A1
(43) Pub. Date: Feb. 19, 2015

(54) METHOD AND SYSTEM FOR TRACING TRAJECTORY OF LESION IN A MOVING ORGAN USING ULTRASOUND

(71) Applicant: Samsung Electronics Co., Ltd., Suwon-si (KR)

(72) Inventors: Young-kyoo Hwang, Seoul (KR); Jung-bae Kim, Hwaseong-si (KR); Won-chul Bang, Seongnam-si (KR); Do-kyoon Kim, Seongnam-si (KR)

(21) Appl. No.: 13/961,057

(22) Filed: Aug. 7, 2013

(51) Int. Cl. A61B 5/06; A61B 19/00

(52) U.S. Cl. CPC A61B 5/06; A61B 19/5244
USPC 600/424

(57) ABSTRACT

A method of tracing a movement trajectory of a lesion in a patient's internal structure, and a system for performing the same. The method includes generating an image showing a movement trajectory of a target portion that is a portion to be irradiated with a therapeutic wave, irradiating the internal structure with a tracking wave, determining a location of the target portion using a reflected wave, and determining whether the location of the target portion is within the movement trajectory.

### CROSS-REFERENCE TO RELATED APPLICATIONS

This application claims the benefit under 35 U.S.C. §119(a) of Korean Patent Application No. 10-2012-0086391, filed on Aug. 7, 2012, in the Korean Intellectual Property Office, the entire disclosure of which is incorporated herein by reference for all purposes.

### BACKGROUND

1. Field

The following description relates to methods and systems for tracing a trajectory along which a target portion, such as a lesion within a patient's organ that is irradiated with ultrasound, moves due to patient's breathing.

2. Description of the Related Art

With the advancement of medical science, techniques for local treatment of tumors have been developed. Such techniques range from invasive surgery, such as open surgery, to minimally-invasive surgery. A recently developed method is a non-invasive surgery using a gamma knife, a cyber knife, and a high-intensity focused ultrasound (HIFU) knife. In particular, the HIFU knife has been widely used in commercial applications as a treatment that is environment-friendly and harmless to the human body.

HIFU treatment includes irradiating a region of a tumor, known as the focal point, with high-intensity focused ultrasound. This causes focal destruction or necrosis of tumor tissue and results in the removal and treatment of the tumor.

HIFU treatment is one of the most widely used methods because it is completely non-invasive; allowing removal of lesions without the need for directly incising a patient's body. However, movements of a lesion resulting from natural physiological processes occurring within a patient's body may cause inaccuracies in targeting the lesion. That is, the location of the lesion may change due to motion within the patient's body making it difficult to accurately account for the position of the lesion during the surgical procedure. For example, when a patient breathes during surgery, the location of a lesion may vary according to the respiratory motion. As the location of the lesion changes, the focal point targeted for irradiation may also need to change. Thus, research is being actively carried out regarding a method for tracking a lesion whose location varies during irradiation.

### SUMMARY

In one general aspect, there is provided a method of tracing a movement trajectory of a lesion in a patient's moving internal structure including generating an image showing a movement trajectory of a target portion that is to be irradiated with a therapeutic wave, irradiating the internal structure with a tracking wave, determining a location of the target portion using a reflected wave, and determining whether the location of the target portion is within the movement trajectory.

The internal structure is a patient's organ, the image may show a movement trajectory of the target portion during one respiratory cycle, the image may be generated based on a plurality of medical images of the patient's organ; and the tracking wave may be a tracking ultrasound wave and the therapeutic wave may be a therapeutic ultrasound wave.

The method may further include irradiating the target portion with the therapeutic wave according to a result of the determining whether the location of the target portion is within the movement trajectory.

The method may further include generating an alarm signal representing cessation of irradiation of the therapeutic wave according to a result of the determining whether the location of the target portion is within the movement trajectory.

The method may further include initiating either one or both of a visual indicator and an auditory indicator in response to the alarm signal.

The method may further include predicting whether a next location of a plurality of locations of the target portion will be within the movement trajectory, and irradiating the target portion with the therapeutic wave according to a result of the predicting.

The generating of the image may include selecting a landmark point from an average model of the organ; and matching the average model of the organ to a medical image of the patient's organ by matching the landmark point to a corresponding location in the medical image.

The image may be a first image, and the generating of the first image may include comprises setting the target portion, indicating the target portion on a second image that demonstrates a movement of the organ and contains unique anatomical information of the patient, and generating the first image using the second image.

The determining of the location of the target portion may include setting a control point at a location in the patient's organ to be irradiated with the tracking ultrasound wave, irradiating the control point with the tracking ultrasound wave and receiving the reflected wave, calculating a location of the control point after the control point moves by using a phase shift of the received reflected wave, and determining a location of the target portion after the target portion moves during one respiratory cycle using the calculated location of the control point.

The method may further include irradiating the internal structure with the therapeutic wave while performing the determining of whether the location of the target portion is within the movement trajectory.

In another general aspect, there is provided a non-transitory computer-readable storage medium storing a program for controlling a computer to execute the method.

In another general aspect, there is provided a system for tracing a movement trajectory of a lesion in a patient's moving internal structure including an irradiation device configured to irradiate the internal structure containing the lesion, a trajectory image generation unit configured to generate an image showing a movement trajectory of a target portion that is a portion to be irradiated with a therapeutic wave, a trajectory information acquiring unit configured to irradiate the internal structure with a tracking wave and determine a location of the target portion using a reflected wave, and a comparator unit configured to determine whether the location of the target portion is within the movement trajectory.

The internal structure may be a patient's organ, the image may show a movement trajectory of the target portion during one respiratory cycle, the image may be generated based on a plurality of medical images of the patient's organ; and the tracking wave may be a tracking ultrasound wave and the therapeutic wave may be a therapeutic ultrasound wave.

The comparator unit may be further configured to generate an alarm signal representing cessation of irradiation of the therapeutic wave according to a result of the determining whether the location of the target portion is within the movement trajectory.

The system may further comprise an image display device configured to initiate either one or both of a visual indicator and an auditory indicator in response to the alarm signal.

The comparator unit may be further configured to predict whether a next location of a plurality of locations of the target portion is within the movement trajectory and transmit a signal indicating irradiation of the target portion with the therapeutic wave to the ultrasound irradiation device according to a result of the predicting.

The trajectory image generation unit may be further configured to select a predetermined landmark point from an average model of the organ and match the average model of the organ to a medical image of the patient's organ by matching the landmark point to a corresponding location in the medical image.

The image may be a first image, and the trajectory image generation unit may include a target portion setting unit configured to set the target portion, and a trajectory marking portion configured to indicate the target portion on a second image that demonstrates a movement of the organ and contains unique anatomical information of the patient.

The trajectory image generation unit may include a control point setting portion configured to set a control point at a location in the patient's organ to be irradiated with the tracking ultrasound wave, a control point location calculator configured to calculate a location after the control point moves using a phase shift of the reflected wave received in response to irradiating the control point with the tracking ultrasound wave, and a target portion location calculator configured to determine a location of the target portion after the target portion moves during one respiratory cycle using the calculated location of the control point.

The system may further include a trajectory image update unit configured to generate a mark indicating the location of the target portion on the image.

### BRIEF DESCRIPTION OF THE DRAWINGS

FIG. 1 is a diagram illustrating an example of high-intensity focused ultrasound (HIFU) system.

FIG. 2 is a diagram illustrating an example of the image processing device in the HIFU system of FIG. 1.

FIG. 3 is a diagram illustrating an example of the organ image generation unit in the image processing device of FIG. 2.

FIG. 4 is a diagram illustrating an example of a method of extracting position coordinates of a boundary and internal structure of an organ in an average model generator.

FIG. 5 is a flowchart illustrating an example of a method of matching a personalized model to a location of an organ in an ultrasound image in a motion image generator.

FIG. 6 is a diagram illustrating an example of a method of acquiring an affine transform function T_{affine} from a two-dimensional (2-D) image.

FIG. 7 is a diagram illustrating an example of a process of matching images to each other in a motion image generator.

FIG. 8 is a graph illustrating an example of a longitudinal movement of a diaphragm's absolute position.

FIG. 9 is a flowchart illustrating an example of an operation of an organ image generation unit.

FIG. 10 is a diagram illustrating an example of a clinical target volume trajectory and a planning target volume trajectory obtained for one respiratory cycle.

FIG. 11 is a diagram illustrating an example of a trajectory image generation unit in the image processing device of FIG. 2.

FIG. 12 is a diagram illustrating an example of a trajectory information acquiring unit in the image processing device of FIG. 2.

FIG. 13 is a diagram illustrating an example of the calculation of displacements of a control point in a control point calculator.

FIG. 14 is a diagram illustrating an example of a reference wave signal and a reflected wave signal.

FIG. 15 is a diagram illustrating an example of information about a location of a target portion for one full respiratory cycle.

FIG. 16 is a diagram illustrating an example of information about a current location of a target portion, which is indicated on an image of a trajectory along which the target portion moves for one respiratory cycle.

FIG. 17 is a diagram illustrating an example of cases in which the current location of a target portion deviates or is expected to deviate from a trajectory along which the target portion moves for a patient's respiratory cycle.

FIG. 18 is a flowchart illustrating an example of operations of a trajectory image update unit and a comparator unit.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

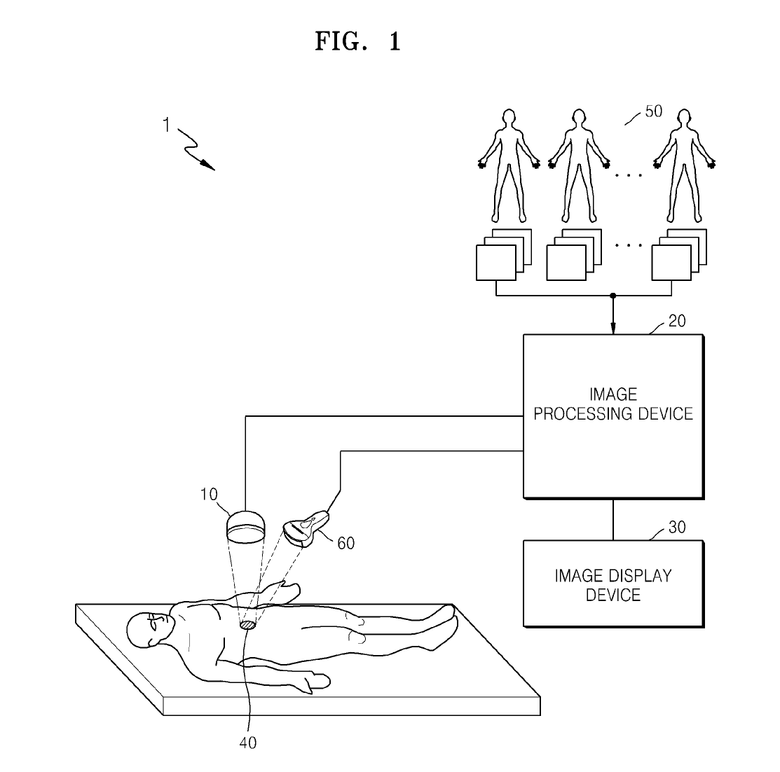

FIG. 1 illustrates an example of a high-intensity focused ultrasound (HIFU) system 1. Referring to FIG. 1, the HIFU system 1 includes an ultrasound therapy irradiation device 10, an ultrasound diagnostic irradiation device 60, an image processing device 20, and an image display device 30. In this example, one or more external medical images 50 of an internal organ containing lesions are gathered from a plurality of people including a patient to be treated. The external medical images 50 may be input to the image processing device 20.

The image processing device 20 uses external medical images 50 and an ultrasound wave that reflects from irradiation by the diagnostic irradiation device 60 of a predetermined point (“control point”) of a lesion 40. The medical images 50 and the reflected wave are used to generate an image showing a trajectory along which a portion (“target portion”) encompassing the lesion 40 moves over one respiratory cycle. An image showing a trajectory of the portion encompassing the lesion as it moves during other physiological cycles, such as a cardiovascular cycle, may also be generated. The method of generating an image showing a trajectory along which the target portion moves will be described in more detail below.

The image processing device 20 also uses an ultrasound wave that reflects from irradiation of a control point by the therapy irradiation device 10. Using the reflected wave, the image processing device may calculate positions of a target portion for one respiratory cycle while a surgical operation is being performed on a patient. The image processing device 20 may then detect and indicate whether the position of the target portion as measured during the surgical operation is consistent with the movement trajectory of the target portion as generated prior to the surgical operation. That is, the image processing device 20 may determine whether the position of the target portion obtained during the surgical operation falls within the preoperative movement trajectory. The image processing device 20 may generate an image showing the movement trajectory and another image showing the position of the target portion during the surgical operation. The two images may be separate images. In another example, the image processing device 20 may generate an image of the movement trajectory indicating the position of the target portion during the surgical operation. This image may be obtained by using a matching technique and thereafter transmitted to the image display device 30.

In this example, the image processing device 20 may generate a predetermined alarm signal when the position of the target portion deviates from or is expected to deviate from the movement trajectory. The alarm signal may be transmitted to the image display device 30 in order to suspend the therapeutic irradiation process. The therapy irradiation device 10 irradiates a predetermined position, i.e., a control point, in a patient's internal organ with the tracking ultrasound. In this example, the therapy irradiation device 10 then receive a reflected wave, and transmits waveform information about the reflected wave to the image processing device 20. More specifically, the therapy irradiation device 10 irradiates a therapeutic ultrasonic wave onto a portion corresponding to the position of a target portion moved over one respiratory cycle of the patient. In an example, the therapy irradiation device 10 may be fixedly installed and may be sufficiently large so as to cover the patient's internal organ including the lesion 40. In alternative examples, the therapy irradiation device 10 may be movable or mobile during the surgical procedure. Although the therapy irradiation device 10 irradiates ultrasound waves downwardly towards the patient, other configurations may be used, such as irradiating ultrasound waves upwardly or in any other direction.

The therapy irradiation device 10 is composed of a plurality of elements for emitting ultrasound. The plurality of elements may receive a signal from a control unit 24 to individually irradiate therapeutic or tracking ultrasound at different times. This construction allows a focal point on which an ultrasound wave converges to change even though the therapy irradiation device 10 is fixedly installed. That is, different positions may be targeted by the ultrasound wave even as the therapy irradiation device 10 remains fixed at one location. Thus, the therapy irradiation device 10 may focus ultrasound waves by tracking lesions that move due to a patient's breathing motion and precisely irradiate a predetermined point with the tracking ultrasound. This technique is called phase array technology.

In an example, super-apertures in the therapy irradiation device 10 may irradiate the therapeutic or tracking ultrasound. In this example, the super-aperture may include a set of some of the elements in the therapy irradiation device 10. Sub-apertures may include a different number of elements. For example, the sub-aperture may include only one element.

Some of the sub-apertures in the therapy irradiation device 10 irradiate the control point with the tracking ultrasound, and the therapy irradiation device 10 may receive a reflected wave. Information about the reflected wave may be transmitted to the image processing device 20. The image processing device 20 obtains a signal indicating movement of a target portion by using the waveform information and transmits the signal to the therapy irradiation device 10.

In this example, the remaining sub-apertures in the therapy irradiation device 10 may irradiate the target portion with a therapeutic ultrasonic wave. In this case, the therapeutic ultrasonic wave may be HIFU having a sufficient energy to cause necrosis of a tumor within the patient's body. The ultrasound therapy irradiation device 10 focuses and irradiates HIFU onto a portion to be treated to cause focal destruction or necrosis of a lesion for removing or treating the lesion. More specifically, when the therapy irradiation device 10 continues to irradiate the focal point by adjusting the focal point of the HIFU to a certain position, the temperature of a cell irradiated with HIFU rises above a predetermined temperature to cause necrosis of surrounding tissue. It will be understood by those of ordinary skill in the art that other ultrasound irradiation devices emitting focused ultrasound similar to HIFU may be used instead of the therapy irradiation device 10 of this example.

The diagnostic irradiation device 60 receives a reflected ultrasonic wave obtained after irradiating the control point with a diagnostic ultrasonic wave. More specifically, when the diagnostic irradiation device 60 irradiates a certain portion with a diagnostic ultrasonic wave in the frequency range of 2 to 18 MHz, the diagnostic ultrasonic wave is partially reflected from different layers of tissue. For example, the diagnostic ultrasonic wave may be reflected from a portion having a density change, such as blood cells within blood plasma or small structures within organs. The reflected diagnostic ultrasonic waves vibrate a piezoelectric transducer of the diagnostic irradiation device 60 that will then output electrical pulses in response to the vibration.

The therapy irradiation device 10 and the diagnostic irradiation device 60 may be separate devices, but are not limited thereto, and may be integrated into a single device or disposed adjacent to each other.

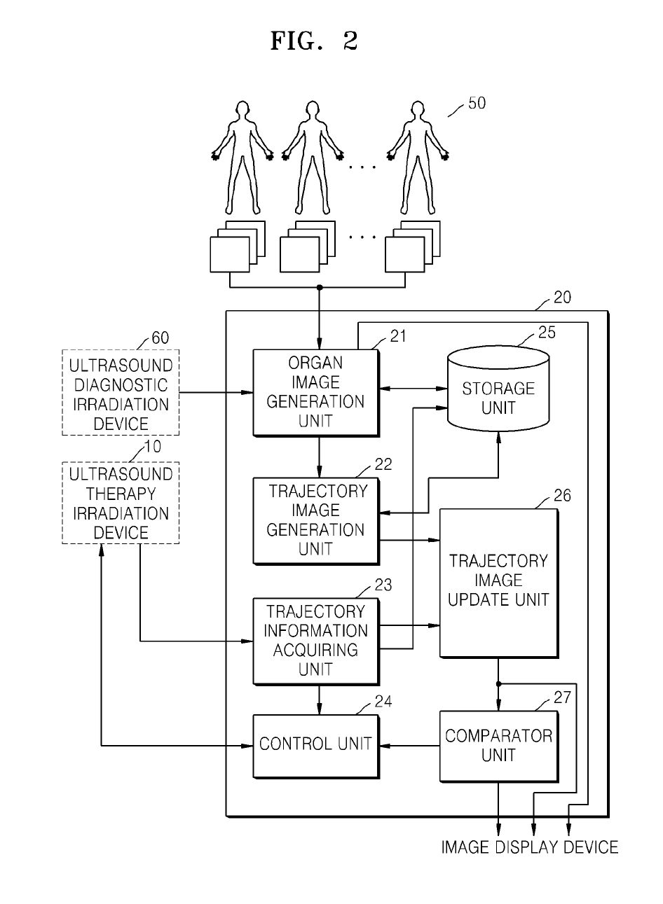

FIG. 2 illustrates an example of the image processing device 20 in the HIFU system of FIG. 1. Referring to FIG. 2, the image processing device 20 includes an organ image generation unit 21, a trajectory image generation unit 22, a trajectory information acquiring unit 23, a control unit 24, a storage unit 25, a trajectory image update unit 26, and a comparator unit 27.

For example, prior to a surgical operation, the organ image generation unit 21 uses one or more medical images 50 of an internal organ containing lesions that are gathered from a plurality of people. In an example, the medical images 50 include at least one medical image of the internal organ containing lesions of the patient to be treated. The medical images 50 show the movement of an internal organ for one respiratory cycle and are used to generate a patient-specific image showing the movement of the internal organ and patient-specific anatomical information. In this example, the diagnostic irradiation device 60 produces the image showing the movement of the internal organ and lesion during one respiratory cycle of the patient by using a reflected ultrasonic wave. As previously explained, the reflected wave is received after irradiating the internal organ with a diagnostic ultrasonic wave from the diagnostic irradiation device 60.

While the organ image generation unit 21 is described as being used for an organ having a lesion, any other anatomical structures having lesions may also be treated. Additionally, while the organ image generation unit 21 may be used for trajectory occurring during a respiratory cycle, the image generation unit 21 may also be used for other physiological cycles causing movements. For example, the image generation unit 21 may trace trajectory during cardiovascular or digestive cycles. The operation of the organ image generation unit 21 will now be described in more detail.

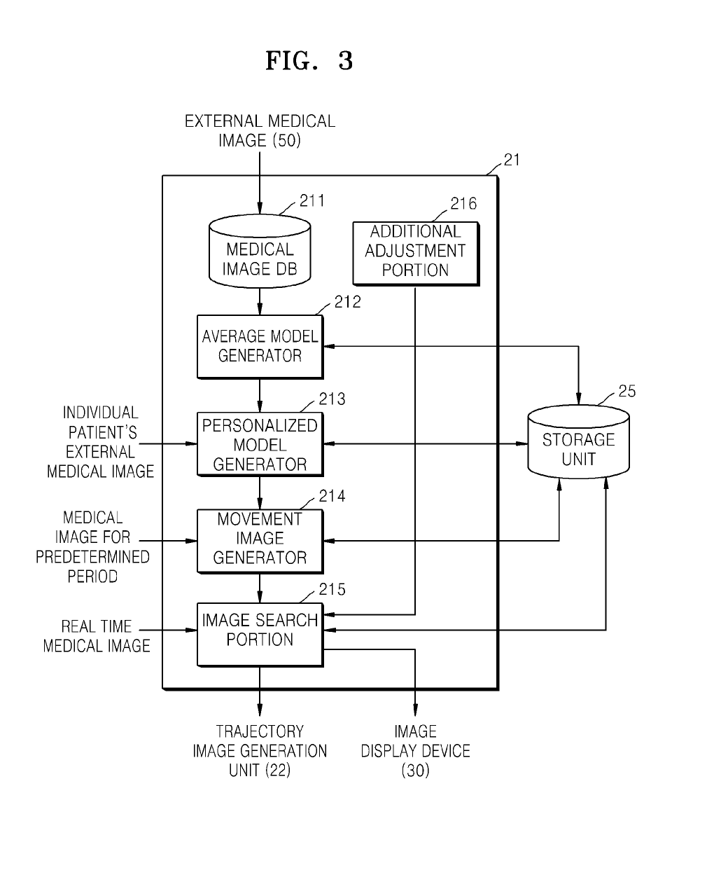

FIGS. 3 through 9 illustrate an example of the generation of an image by the organ image generation unit 21. The generated image shows the movement of an internal organ and contains patient-specific anatomical information. Generation of a 3-D organ model or matching between the 3-D organ model and ultrasound images are not limited to the following description and may be performed in other various manners.

FIG. 3 illustrates an example of the organ image generation unit 21. Referring to FIG. 3, the organ image generation unit 21 includes a medical image database (DB) 211, an average model generator 212, a personalized model generator 213, a movement image generator 214, an image search portion 215, and an additional adjustment portion 216.

In this example, the average model generator 212 receives the medical image or images 50 related to an organ including lesions of a plurality of people. After receiving the one or more images 50, the average model generator 212 processes the external medical images 50, and outputs an average model of the organ containing anatomical information. In this example, a patient's personalized model is generated to track the movement of the organ to be treated. The average model generator 212 may generate an average model as a preparatory step to producing a personalized model. That is, since characteristics such as the shapes, dimensions, and properties of organs are different for each individual, characteristics of each patient need to be reflected in order to allow for a more precise surgical procedure. Various personal image information may be used to obtain an accurate average model. This personal image information may be obtained from various patients previously treated or random individuals not previously treated. Furthermore, images obtained from each individual may include images obtained at various points of a breathing cycle in order to reflect the shape of an organ that changes due to breathing motion.

In this example, the average model generator 212 receives the external medical image or images 50 directly from a photographing apparatus or a storage medium having images stored thereon. The average model generator uses the one or more images 50 in order to analyze the shapes and dimensions of organs in different individuals. Thus, the input images may have defined contours of organs and lesions to allow easy analysis of the characteristics of internal anatomical structures. For example, computed tomography (CT) or magnetic resonance (MR) images may be input as the external medical images 50. In another example, the medical images may be ultrasound images of patients previously treated using the HIFU system 1.

The external medical images 50 may be input by retrieving image data stored in the medical image DB 211. The medical image DB 211 may have stored thereon the external medical images 50 related to different individuals that are directly captured by a photographing apparatus or input from a storage medium. All images, or some images (according to a user's selection) may be retrieved from the medical image DB 211.

In this example, the average model generator 212 may apply a 3-D active shape model (ASM) algorithm based on the input external medical images 50. In order to apply the ASM algorithm, the average model generator 212 may analyze the external medical images 50 to extract the shape, dimensions, and anatomical features therefrom. The average model generator 212 may also average the shape, the dimensions, and anatomical features to produce a statistical average model. The ASM algorithm is described in detail in the paper, “The Use of Active Shape Models For Locating Structure in Medical Images” presented in 1994 by T. F. Cootes, A. Hill, C. J. Taylor and J. Haslam. The ASM algorithm is used to derive the mean shape of an internal organ using the one or more medical images 50. The algorithm allows the mean shape that is derived to be deformed according to adjustments to one or more different variables. In an example, the adjustments made to the one or more different variable may be patient-specific adjustments allowing the generation of a more accurate patient-specific organ model.

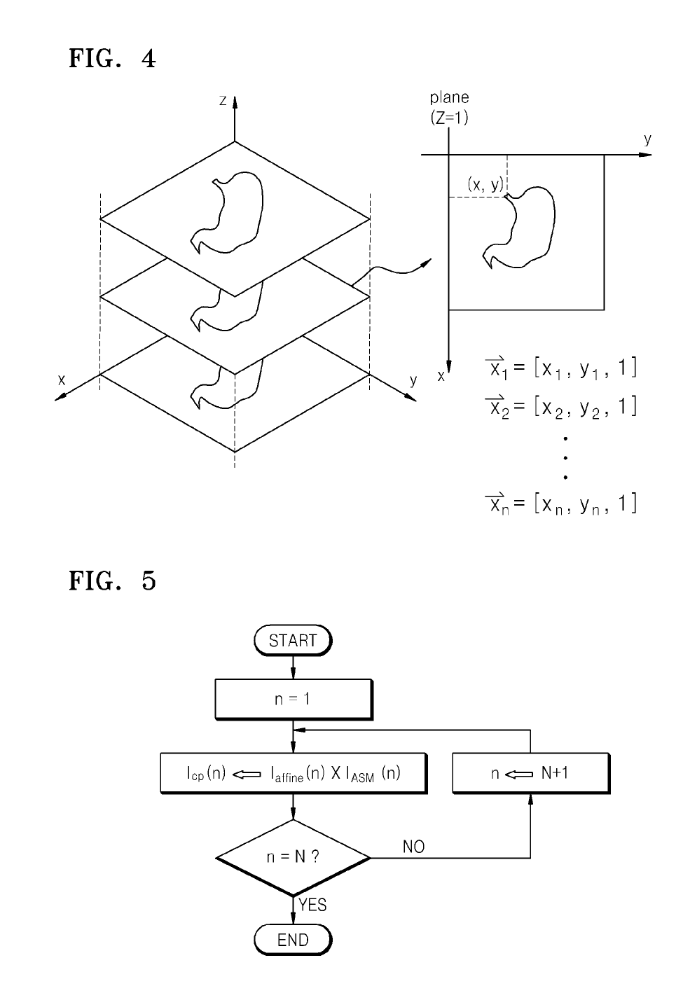

FIG. 4 illustrates an example of a method of analyzing the one or more medical images 50 by extracting position coordinates of a boundary and internal structure of an organ from an input CT or MR image. Upon receipt of the CT or MR image, the average model generator 212 may extract position coordinates in different manners depending on whether the input image is a 2-D or 3-D image. For example, when the organ is a patient's liver, the internal structures that are analyzed may include a hepatic artery, a hepatic vein, a hepatic portal vein, a hepatic duct, and boundaries between them.

In an example where a 2D image is input, in order to generate a 3D model, the average model generator 212 accumulates a plurality of 2D cross-sectional images and obtains 3D volume image data. That is, 3D volume image data corresponding to a 3D region of interest may be obtained using a plurality of 2D cross-sectional images. The process of accumulating a plurality of image data and obtaining a 3-D volume image is shown on the left side of FIG. 4. In this example, the average model generator 212 extracts the position coordinate information of the boundary and the internal structure of the organ from each of the plurality of cross-sectional images before accumulating the plurality of cross-sectional images and before obtaining a 3D image. In order to obtain 3D coordinate information using the extracted 2D coordinates, the average model generator 212 adds coordinate information of an axis having a direction in which the cross-sectional images were accumulated. As shown on the right side of FIG. 2, if the 2D cross-sectional area is in the XY plane, a Z value for the position coordinates extracted from a first image would always be 1. Thereafter, a Z value for the position coordinates extracted from a second image of a cross-section above the first cross-sectional area would always be 2. For the example shown on the right side of FIG. 2, 2D coordinate information represented by a coordinate pair [x,y] and a Z-axis coordinate value are combined to obtain the 3-D position coordinate information [x,y,1] of the boundary. Thus, in this example, the obtained coordinate information is 3D coordinate information including x, y, and z coordinates.

In an example where a 3D image is input as the external medical image 50, the average model generator 212 may first extract cross-sections of the 3D image at predetermined intervals and then performs the same process as when the 2D image is input. The result is obtaining 3D position coordinate information. The coordinate information may be of the boundary and the internal structure of an organ.

Position coordinates of the boundary of an organ in a 2D image may be automatically or semi-automatically obtained by using an algorithm. Alternatively, the position coordinates may be manually input by a user based on the image. For example, in a method of automatically obtaining the position coordinates of the boundary of the organ, the position coordinates of the boundary may be coordinate information of a portion of the image in which the brightness changes abruptly. That is, in detecting the region where brightness changes abruptly, the average model generator 212 automatically detects the boundary of an organ, and may then extract the position coordinators corresponding thereto. Alternatively, a location where a frequency value is the highest may be detected as the boundary of an organ. Coordinate information corresponding to such a region may be automatically extracted by using discrete time Fourier transformation (DTFT).

In a method of semi-automatically obtaining the position coordinates of the boundary of the organ, if information about some boundary points of an image is input by a user, the coordinates of the entire boundary may be extracted in the same way as in the method of automatically obtaining the position coordinates. Since the boundary of the organ has a continuous closed curve shape, information about the entire boundary of the organ may be obtained. The semi-automatic method eliminates the need for searching the whole image, thereby obtaining the position coordinates of the boundary quicker than in the automatic method.

In a method of manually obtaining the position coordinates of the boundary of the organ, a user may directly designate coordinates of a boundary while viewing the image. In this example, since an interval at which the coordinates of the boundary is designated may not be continuous, the coordinates of the boundary may be extracted continuously by performing interpolation with respect to discontinuous intervals.

Once the position coordinates of the 3D region are obtained using the example methods described above, the user may view the shape of the organ and internal structure using 3-D graphics. The brightness value of a voxel corresponding to the extracted position coordinates nay be set to a predetermined brightness level. For example, if a brightness value of boundary coordinates of a target organ is set to a minimum level, i.e., the darkest value, an image of the target organ is output as black. If the brightness value of the target organ is set to a medium level between white and black, and the brightness value of a lesion is set to black, the target organ may easily be distinguished with the naked eye from the lesion. In this example, the extracted position coordinate information of the boundaries and structures for one or more organs of different individuals may be defined as a data set and used to carry out the ASM algorithm as described below.

In order to apply the ASM algorithm, axes of position coordinates of the boundaries and structures of the plurality of organs are fitted to one another. Fitting the axes to one another means placing the center of gravity of the plurality of organs at the same origin. Also, fitting the organs to one another may include aligning the directions of the plurality of organs having various forms to one another. Thereafter, landmark points are determined in the position coordinate information of the boundaries and the internal structures of the plurality of organs. In this example, landmark points are basic points for applying the ASM algorithm. The landmark points may be determined using the following method.

First, coordinate points corresponding to distinctive regions of a target object are determined as landmark points. For example, where the target object includes the liver, such points may correspond to a division of a blood vessel that everyone has in their liver. In an example where the target object includes the heart, such points may correspond to a boundary between left and right atria in a heart or a boundary at which a main vein and an outer wall of the heart meet. Various other examples of distinctive regions in target objects may also be included.

Second, points corresponding to the highest and lowest points of a target object are also determined as landmark points. For example, the highest and lowest points of an the liver or the heart may be determined as landmark points.

Third, points that may be used for interpolating a space between the first set of landmark points and the second set of landmark points may also be determined as landmark points. Such points may exist along a boundary at predetermined intervals.

In this example, the determined landmark points may be represented by x and y coordinates in a 2D image and by x, y, and z coordinates in a 3D image. Thus, when coordinates of each landmark point are indicated by vectors x₀, x₁, . . . xₙ, in a 3D image (where n is the number of landmark points), the vectors x₀, x₁, . . . xₙ, may be defined by Equation (1):

$${x}_{i 0} = [ {x}_{i 0} , {y}_{i 0} , {z}_{i 0} ] \\ {x}_{i 1} = [ {x}_{i 1} , {y}_{i 1} , {z}_{i 1} ] \\ ⋮ \\ {x}_{i n - 1} = [ {x}_{i n - 1} , {y}_{i n - 1} , {z}_{i n - 1} ] \qquad ( 1 )$$

The subscript i indicates position coordinate information in an i-th image. When the position coordinate information is increased, the position coordinate information may be represented by a single vector to facilitate calculation. That is, a landmark point vector may represent all of the landmark points as defined by Equation (2):

xᵢ=[xᵢ₀, yᵢ₀, zᵢ₀, xᵢ₁, yᵢ₁, zᵢ₁, . . . , xᵢₙ₋₁, yᵢₙ₋₁, zᵢₙ₋₁]^{T} (2)

The magnitude of the vector xᵢ is 3n×1. If the number of images in the data set is N, an average of the landmark points for all of the images in the data set is defined by Equation (3):

$$\overline{x} = \frac{1}{N} {\sum }_{i = 1}^{N} {x}_{i} \qquad ( 3 )$$

Likewise, the magnitude of the vector x̅ is 3n×1. In this example, the vector x̅ is a calculated average of the plurality of landmark vectors describing organs of different patients. The average model generator 212 obtains the average x̅ of the landmark points by using Equation 3, and generates an average model for an internal organ based on the average vector x̅. The ASM algorithm is used to generate the average organ model. Additionally, once the average organ model is generated, the ASM algorithm may be used to deform or alter the average organ model by adjusting a plurality of parameters. Thus, as well as being able to calculate the average organ model, the average model generator 212 uses an equation to apply the plurality of parameters. An equation for applying the plurality of parameters will be described below.

A difference between each landmark vector xᵢ and the average landmark vector x̅ is calculated using Equation 4. The subscript i denotes an i-th image. Thus, Equation 4 indicates a difference between the landmark points xᵢ in an ith image and the average x̅ of the landmark points.

dxᵢ=xᵢ− x̅ (4)

A covariance matrix S for three variables x, y, and z may be defined by Equation 5. The size of the covariance matrix S is 3n×3n, as described in equation 5 below. The covariance matrix S is used to obtain a unit eigenvector with respect to the plurality of parameters for applying the ASM algorithm. This has been described in detail in the above-mentioned paper, “The Use of Active Shape Models For Locating Structure in Medical Images”.

$$S = \frac{1}{N} {\sum }_{\text{?} = 1}^{N} d {x}_{\text{?}} d {{x}_{\text{?}}^{\text{?}}}_{\text{?}} 3 n \times 3 n ) \\ \text{?} \text{indicates text missing or illegible when filed} \qquad ( 5 )$$

If the unit eigenvector of the covariance matrix S is Pₖ (k=1, 2, . . . 3n), the unit eigenvector Pₖ corresponds to modes of variation of the average organ model generated by using the ASM algorithm. For example, if a parameter b₁ multiplied by a vector P₁ changes within a range of −2\sqrt{λ₁}≦b₁<2\sqrt{λ₁}, a width of the model may be changed. If a parameter b₂ multiplied by a vector P₂ changes within a range of −2\sqrt{λ₂}≦b₂<2\sqrt{λ₂}, a height of the model may be changed. The unit eigenvectors having a magnitude of 3n×1 may be obtained by using Equation (6):

Spₖ=λₖpₖ (6)

where λₖ denotes an eigen-value.

Finally, the landmark point vector x in which the variation of the model is reflected may be calculated by using the average vector x̅ of the landmark points as in Equation (7):

x= x̅+Pb (7)

p=(p₁, p₂, . . . pₜ) denotes first t unit eigenvectors (the magnitude of unit eigenvector pₖ is 3n×1, and the magnitude of P is 3n×t), and (with a magnitude of t×1) denotes weights of each eigenvector, b=(b₁, b₂, . . . bₜ)^{T}

The average model generator 212 uses the above-described Equations to calculate x̅ (with a magnitude of 3n×1) representing the shape of an average organ model and the vector p=(p₁, p₂, . . . pₜ) (with a magnitude of 3n×t) which is used to apply the variation of the model by using the 3D ASM algorithm.

In this example, the personalized model generator 213 receives the average model x̅ for an internal organ and the vector p=(p₁, p₂, . . . pₜ) (with a magnitude of 3n×1) from the average model generator 212. The personalized model generator 213 may then generate a patient's personalized model for the organ through parameter processing of the ASM algorithm. Since shapes and dimensions of organs are different for each patient, the use of a personalized model ensures a more accurate model. For example, an organ of a patient may be wider, longer, or thicker on the left side. An organ of a patient may also fall more on a right side than the average organ shape. In addition, if a patient's organ includes a lesion, the personalized model generator 213 may generate a model including an accurate shape and location of the lesion. Thus, the personalized model generator 213 receives the one or more external medical images 50 of the individual patient from an image photographing apparatus or a storage medium, and analyzes a shape, dimension, and location of the patient's organ. Also, if a lesion is detected in the one or more images 50, the personalized model generator 213 also analyzes a shape, dimension, and location of the lesion. The operation of the personalized model generator 213 will now be described in detail.

In this example, the personalized model generator 213 determines weights (the vector b) of a unit eigenvector used in the ASM algorithm for the individual patient based on medical images such as CT or MR images clearly demonstrating the shape of an organ. For example, the personalized model generator 203 may first receive an external medical image 50 of the individual patient and obtain position coordinate information of a boundary and an internal structure of an organ. To obtain position coordinate information, the personalized model generator 203 may analyzes the external medial image 50 by using the same process described above for the average model generator 212 as illustrated in FIG. 4.

Furthermore, by determining coordinate information of landmark points using the same process, a vector x (with the magnitude of 3n×1), which is a set of personalized landmark points, may be obtained. A personalized model may be built by generating an organ model based on the vector x. An organ model generated based on the vector x may be a private model. Properties of a unit eigenvector and an inverse function (pₖ^{T} pₖ=1) are applied to Equation (7) to create the following Equation (8). A value of b=(b₁, b₂, . . . bₜ)^{T} is determined by Equation (8):

b=P^{T} (x− x̅) (8)

In an example, information about the vectors x̅ and p that are determined by the average model generator 212 may be stored in the storage unit 25 as a database of an average model for each iteration. Furthermore, the external medical image 50 of the individual patient that is input to the personalized model generator 213 may be used as a training set in determining an average model during a future medical examination of another patient.

After the personalized model generator generates a patient-personalized model, a movement image generator 214 may generate an image indicating the movements resulting from a physiological cycle over a predetermined period of time. For example, upon receipt of the vectors x, x̅, p, and b from the personalized model generator 213, the movement image generator 214 matches the vectors x, x̅, p, and b to a medical image of the patient obtained during a predetermined respiratory cycle. This matching means that a model generated using the ASM algorithm is superimposed on a location of an organ in an ultrasound image to output a superimposed image. In other words, pixel or voxel values corresponding to coordinate information of a model obtained using the ASM algorithm replace or overlap the organ of the ultrasound image. The pixel or voxel values may have a predetermined brightness. When the pixel or voxel values replace the organ of the ultrasound image, the organ is removed from the original ultrasound image and only a personalized model is output.

In another example, pixel or voxel values of the model may simply overlap the organ of the ultrasound image, and an image in which the personalized model is superimposed on the original ultrasound image may be output. In this example, the superimposed image may be easily distinguished with the naked eye by using different colors. For example, a blue-colored personalized model may be superimposed on a black and white ultrasound image so as to easily distinguish its shape from the organ of the original ultrasound image.

The medical image obtained during a physiological cycle, such as a respiratory cycle, may include the movement of the patient's internal organ and a lesion. For example, the medical image may be a 2D or 3D ultrasound image. The predetermined cycle for which the image corresponds to may be one cycle because an organ may change in shape during only one physiological cycle. In this example, a physiological cycle may include a body's respiratory cycle; however, other cycles may include a cardiovascular or digestive cycle.

In an example, the process of matching the 3D model and medical image by the movement image generator 214 may be divided into two operations. The first operation includes reflecting in the 3D organ model a change of an organ due to breathing as shown in an input ultrasound image during the predetermined period. The second operation may include aligning the 3-D organ model, including the reflected changes, to the target organ in the ultrasound image by scaling, rotating, and translating.

In this example, the first operation is reflecting a change of the organ due to breathing in the 3D organ model. Before matching the 3D organ model to the medical image, the movement image generator 214 adjusts a value of the vector b of weights for each unit eigenvector, which is a parameter for the ASM algorithm, by detecting a location and a change of an organ for each frame of the ultrasound image. It should be appreciated that the determined value of the vector b should generally not have a large deviation from the value of the vector b obtained by the average model generator 212. This is because the movement image generator 214 reflects only a change due to the breathing, which is trivial between individuals. Thus, in determining the value of vector b, the value of vector b determined by the average model generator 212 may be taken into consideration, and the change made to the organ model may fall within predetermined limits based on vector b of the average model generator 212. Additionally, the value of a vector b for a previous frame may be used to determine the value of a vector b for the following frame because a change of an organ during a breathing motion is continuous and may include small changes that occur quickly during short frames. When the value of vector b is determined, it is possible to generate a personalized model for each frame, in which the change of the organ is reflected in each ultrasound image by using operations of the 3D ASM algorithm.

FIG. 5 is a flowchart illustrating an example of a process in which the movement image generator 214 fits a personalized model including reflected changes in each image to a location of the organ in an ultrasound image through rotation, scaling, and translation. Referring to FIG. 5, this process may be performed by one-to-one affine registration for each frame. Affine registration may be completed when the vector b, indicating the weights of each unit eigenvector, is determined for each frame. When the number of frames is N and n is a frame number, one-to-one matching is performed on first through N-th frames. In this example, an affine transform function T_{affine} is obtained by applying an iterative closest point (ICP) algorithm for each frame. The ICP algorithm may use a landmark point set of the ultrasound image and a landmark point set of the model to obtain T_{affine}. A 3D body organ model may then be transformed using the affine transform function T_{affine}. The ICP algorithm is employed for aligning a target object within a plurality of images. For example, the target object within the plurality of images may be rotated, translated, and scaled with respect to one of the plurality of images. The ICP algorithm is described in detail in the paper, “Iterative Point Matching for Registration of Free-form Curves and Surfaces,” presented by Zhengyou Zhang, which is incorporated herein by reference in its entirety.

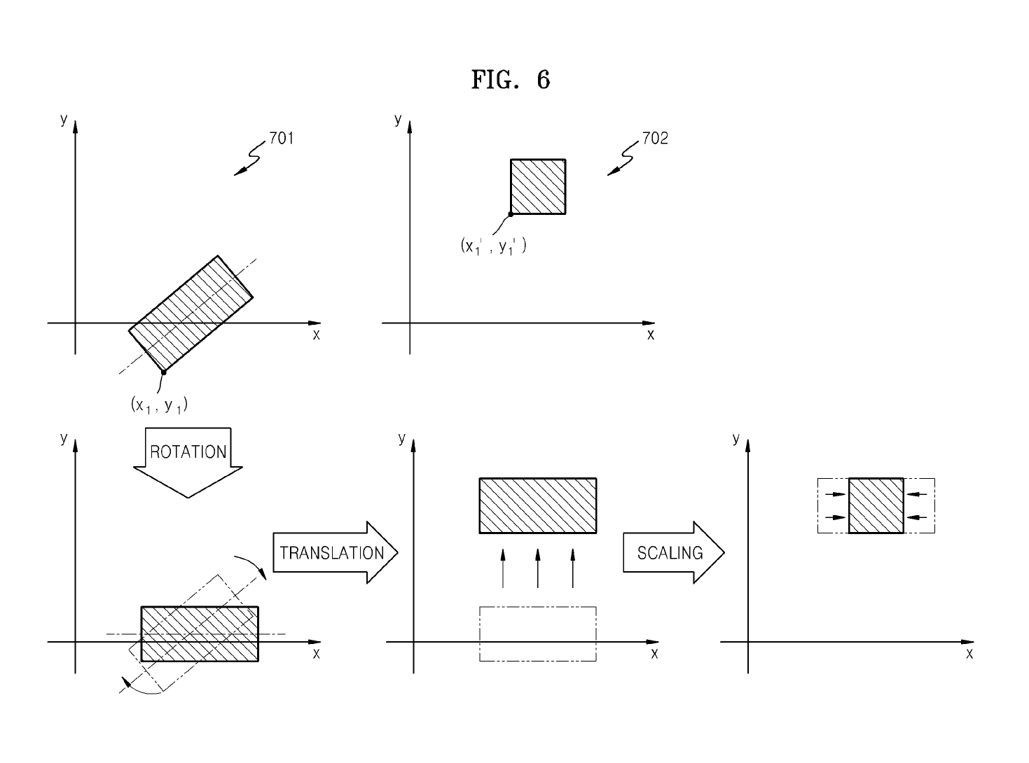

FIG. 6 illustrates an example of a method of acquiring an affine transformation function T_{affine} from a 2D image. Reference numeral 701 represents a state of an object before the affine transformation is applied, and reference numeral 702 represents a state of the object after the affine transformation is applied. Although rotation, translation, and scaling is performed by the affine transformation, first coordinates and last coordinates may be obtained using Equation (9) to determine coefficients of a matrix T_{affine}. In this example, first coordinates refer to coordinate points prior to the transformation and last coordinates refer to coordinate points after the transformation. Since the affine transform uses a one-to-one point correspondence, obtaining first and last coordinates allow the determination of transformation matrix T_{affine} as shown in Equation (9) below.

$$[ \begin{matrix}{x}_{1}^{′} \\ {y}_{1}^{′}\end{matrix} ] = {T}_{affine} [ \begin{matrix}{x}_{1} \\ {y}_{1} \\ 1\end{matrix} ] = [ \begin{matrix}{a}_{1} & {b}_{1} & {c}_{1} \\ {a}_{2} & {b}_{2} & {c}_{2}\end{matrix} ] [ \begin{matrix}{x}_{1} \\ {y}_{1} \\ 1\end{matrix} ] \qquad ( 9 )$$

Equation (10) is used for applying the affine transformation function T_{affine}, obtained in three and more dimensions, to each frame:

x_{ICP}(n)=T_{affine}(n)×x_{ASM}(n) (10)

wherein n is an integer indicating an n-th frame (1≦n≦N), and x_{ASM}(n) denotes a landmark point vector obtained by changing the vector b of weights in the movement image generator 214.

x_{ICP}(n) includes position coordinates of boundaries and internal structures of an organ in which a variation is reflected for each frame. When the postion coordinates are matched to the ultrasound image, voxel values corresponding to the position coordinates may be replaced or may overlap the existing values with a predetermined brightness value. This allows recognizing the shape of the organ with the naked eye.

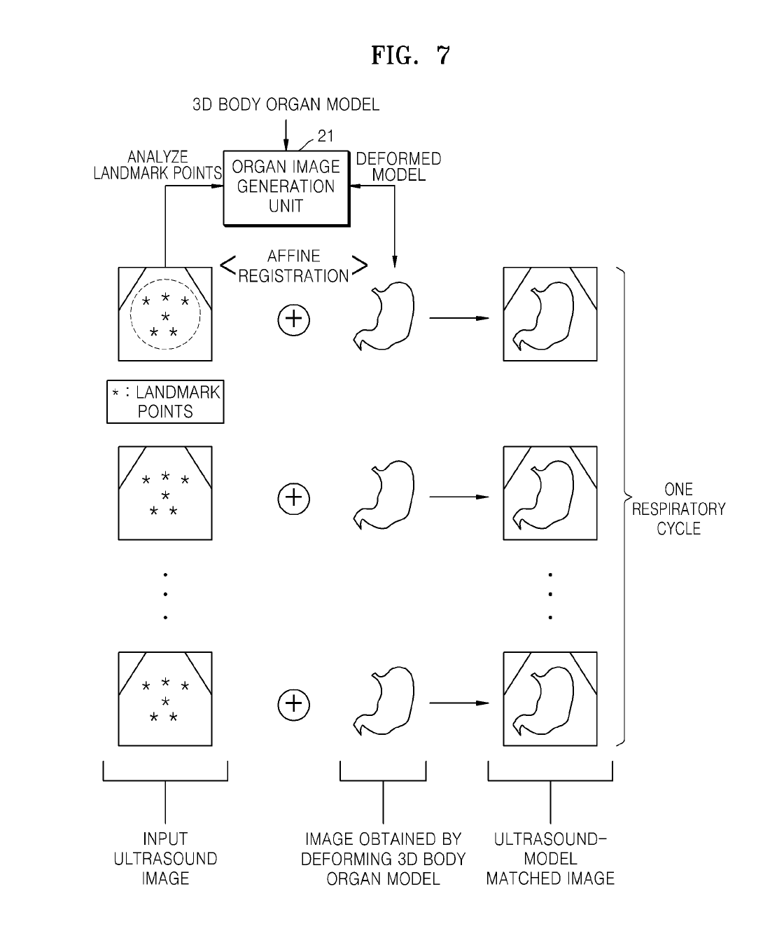

FIG. 7 illustrates an example of a process of matching images to each other in the motion image generator 214. Referring to FIG. 7, the image matching unit 214 matches a personalized 3-D organ model to a plurality of input ultrasound medical images for one respiratory cycle. The input ultrasound images including landmark points are disposed on the left side of FIG. 7. As shown in the figure, ‘*’ in the input ultrasound images indicates landmark points. The input ultrasound images may reflect various types of breathing from inspiration to expiration.

The shape of a personalized 3D body organ model as generated by the personalized model generator 213 may then be deformed according to the patient's breathing motion. However, the deformation due to breathing may be smaller than deformation due to diversity between individuals. Thus, as a method of reflecting a variation in the personalized model due to breathing, adjusting parameter values determined by the personalized model generator 213 may be faster and easier than calculating new parameter values using the ASM algorithm method previously described. The affine transform function T_{affine} is applied through an ICP algorithm by using landmark points of the model, in which the variation has been reflected, and landmark points of the organ in the ultrasound image. An affine transformation is applied to deform dimensions and locations of the personalized 3D organ model to better fit dimensions and locations of an organ in the ultrasound image. Combining the deformed model with the ultrasound image may be accomplished by replacing or overlapping a pixel or voxel value in the ultrasound image. A matched image is referred to as an ultrasound-model matched image and may be stored in the storage unit 25.

In this example, the organ image generation unit 21 also includes an image search portion 215. The image search portion 215 performs processing during a surgical operation. During a surgical operation, an ultrasound image graphically displays the shape of an organ in real time on a screen, and a surgeon performs the surgical operation while viewing the ultrasound image. For example, first, a real time medical image of a patient is received. In this example, the real time medical image may be the same image as that received by the movement image generator 214, i.e., an ultrasound image. The input real time ultrasound image may then be compared with the medical images that were input to the movement image generator 214 for a predetermined period. A medical image that is the most similar to the real time ultrasound image is determined by comparing the images. The storage medium 25 may then be searched for an ultrasound-model matched image corresponding to the determined medical image that was input to the movement image generator 214, and a found ultrasound-model matched image is output.

When the image search portion 215 searches for an image that is similar to the real time ultrasound image, the similar medical image may be determined by detecting a location of a diaphragm. For example, if the diaphragm is located at point X in the real time ultrasound image, the image search portion 215 may calculate a difference between the point X in each of the plurality of medical images, which are input to the movement image generator 214, and the actual location of the diaphragm in each of the images for the predetermined period. The image search portion 215 may then search for an image having the smallest difference in order to find the image that is most similar to the real time ultrasound image.

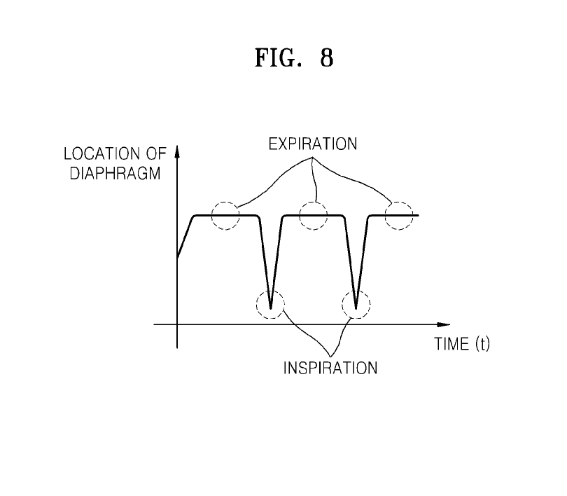

FIG. 8 is a graph illustrating an example of a longitudinal movement of a diaphragm's absolute position. As is evident from the graph, the position of the diaphragm changes regularly during each respiratory cycle, changing its position over time during expiration and inspiration. A location of the therapy irradiation device 10 and a patient's location may be fixed during capturing of the medical images which are input to the movement image generator 214. Similarly, the location of the therapy irradiation device 10 and the patient's location may be fixed during capturing of the real time medical image that is input to the image search portion 215. However, any change in the location of the therapy irradiation device 10 with respect to the patient, or any movement by the patient with respect to the therapy irradiation device 10, may induce a change in the relative location of an organ. This makes it difficult to accurately and rapidly perform a search upon comparing images.

In another example, the image search portion 215 may search for an image that is similar to the real time ultrasound image by using a brightness difference between pixels. This method uses the principle that the most similar images have the smallest difference in brightness levels. More specifically, when searching the medical images (“first images”) for an image that is most similar to an image of a frame in the real time ultrasound (“a second image”), the image search portion 215 may calculate a brightness difference between each pixel of one of the first images and each pixel of the second image to obtain a dispersion for the brightness differences. The image search portion 215 may also compare the dispersions for brightness differences between pixels of the remaining first images and pixels of the second image in the same manner to determine the image having the smallest dispersion as the most similar image.

In this example, the organ image generation unit 21 also includes an additional adjustment unit 216. The additional adjustment unit 216 allows a user to adjust the output image by adjusting parameters for the affine transform function T_{affine} and the ASM algorithm while viewing the output image. That is, the user may perform accurate transformation or adjustment to the output image while viewing the image on the image display device.

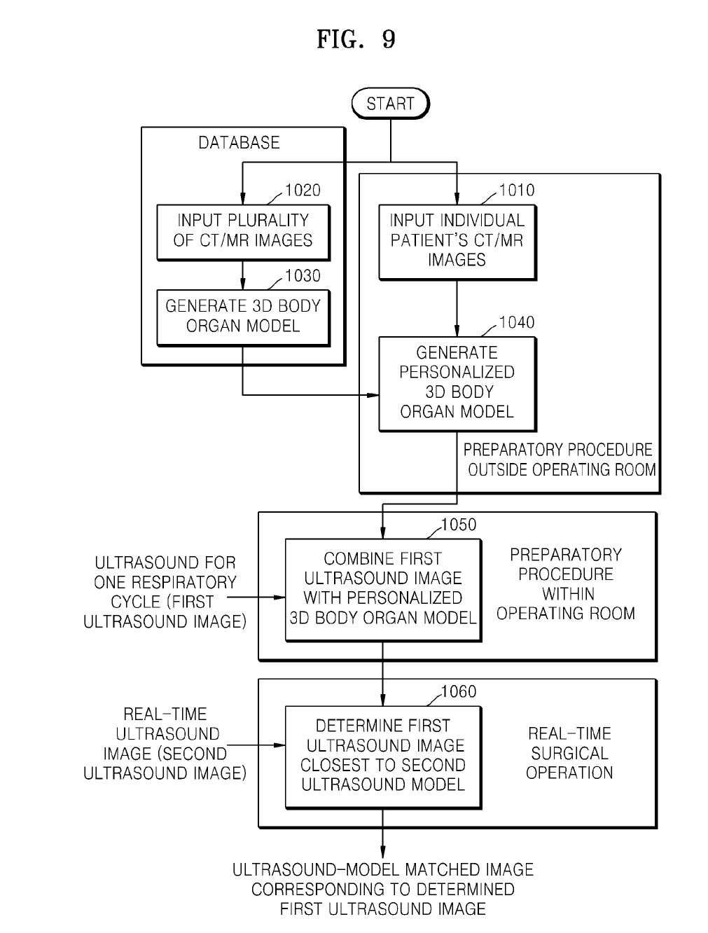

FIG. 9 is a flowchart illustrating an example of an operation of the organ image generation unit 21. A plurality of CT or MR images for various respiratory cycles of each individual are received (operation 1020). A personalized 3D body organ model is generated based on the received images by using an ASM algorithm as described above (operation 1030). An individual patient's CT or MR images are received (operation 1010). The personalized 3D body organ model is generated in operation 1030, and the personalized model is deformed based on the received CT or MR images (operation 1040). The personalized model may be generated outside an operating room as a preparatory process. Ultrasound images for a patient's respiratory period (“first ultrasound images”) are received and matched to the personalized 3D body organ model (operation 1050). The matched image is called an “ultrasound-model matched image” and may be stored in a temporary memory or storage medium such as the storage unit 25. Operation 1050 may be performed as a preparatory process within the operating room. The location of a patient and the therapy irradiation device 10 may be fixed in operations 1050 and 1060. In operation 1060, which is performed in the operating room in real time, a patient's real time ultrasound image (“second ultrasound image”) is received. One of the first ultrasound images that is the most similar to the second ultrasound image is determined. An ultrasound-model matched image corresponding to the determined first ultrasound image is generated. The generated ultrasound-model matched image shows the movement of the internal organ for one respiratory cycle and contains patient-specific anatomical information.

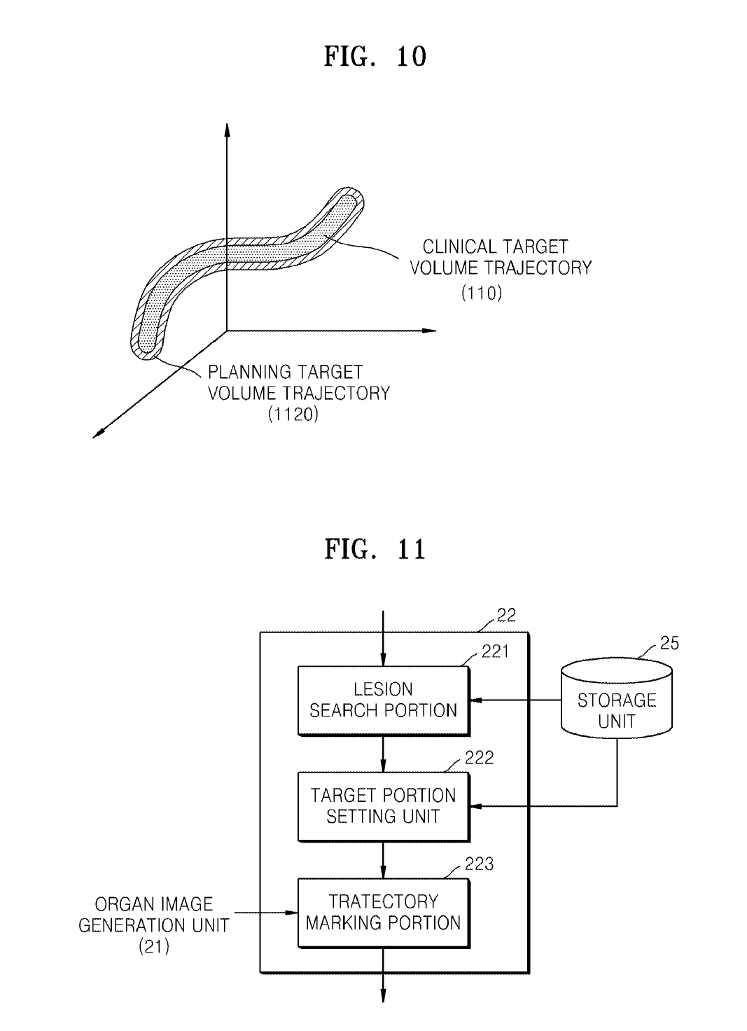

FIGS. 10 and 11 illustrate an example of a method for generating an image showing a trajectory along which a target portion moves during a patient's respiratory cycle.

In order to treat a cancerous lesion by HIFU irradiation, a target portion surrounding a region where the cancerous cells are located is irradiated with HIFU to cause necrosis of the cancer cells. HIFU irradiation of the target portion, which surrounds the cancer cells, may increase the effectiveness of treatment by reducing the chance of transferring cancer cells to different organs. However, there may be an error between a location of cancer cells detected through irradiation of a tracking ultrasound and the actual location of the cancer cells. Accordingly, in this example, a predetermined portion including a location of a lesion previously detected may be selected as a target portion, and movement of the target portion may be accurately monitored during irradiation.

For example, an internal organ including a lesion iteratively moves along a predetermined trajectory due to a patient's breathing motion. Referring to FIG. 10, as a lesion (clinical target volume) moves along a predetermined trajectory 1110, a target portion to be irradiated moves along a predetermined trajectory 1120 corresponding to the trajectory 1110 of the lesion.

In this example, the image processing device 20 of FIG. 2 includes a trajectory image generation unit 22. The operation of the trajectory image generation unit 22 for generating an image showing a movement trajectory of a target portion during one respiratory cycle will now be described in detail with reference to FIGS. 10 and 11.

FIG. 11 illustrates an example of a trajectory image generation unit 22. Referring to FIG. 11, the trajectory image generation unit 22 includes a lesion search portion 221, a target portion setting unit 222, and a trajectory marking portion 223.

The lesion search portion 221 receives the ultrasound-model matched image generated by the organ image generation unit 21. The ultrasound-model matched image shows the movement of the internal organ for one respiratory cycle and contains patient-specific anatomical information. The lesion search portion 221 searches the received image for a location of a lesion, and transmits information about the found location of the lesion to the target portion setting unit 222.

In this example, the image received from the organ image generation unit 21 includes anatomical information of the organ containing the lesion. The lesion search portion 221 may distinguish normal tissue from the lesion based on the anatomical information of the organ. More specifically, the lesion search portion 221 may distinguish the normal tissue from the lesion due to the color difference and blood vessel distribution between the lesion and normal tissue. Also, the storage unit 25 may transmit stored information about differences between the lesion and normal tissue to the lesion search portion 221. This allows the lesion search portion 21 to search for the location of the lesion using the received information. Furthermore, a surgeon may directly search for the location of the lesion while viewing the ultrasound-model matched image on the image display device 30.

The target portion setting unit 222 receives the information about the location of the lesion from the lesion search portion 221 and designates a portion surrounding the lesion as a target portion. That is, the target portion setting unit 222 designates a portion including the lesion and a surrounding region as the target portion to be irradiated with an HIFU; this increases the effectiveness of HIFU treatment.

In this example, the target portion may be determined based on error information stored in the storage unit 25. Error information indicates an error between the location of cancer cells detected through the tracking ultrasound and the actual location of the cancer cells. The storage unit 25 transmits the error information to the target portion setting unit 222, and the target portion setting unit 222 uses the error information to set a target portion to be irradiated by a therapeutic ultrasonic wave of the therapy irradiation device 10. Alternatively, the surgeon may directly determine the target portion to be irradiated, based on the state of the lesion and the error information, by viewing the ultrasound-model matched image through the image display device 30.

The trajectory marking portion 223 uses the target portion set by the target portion setting unit 222 and the ultrasound-model matched image generated by the organ image generation unit 21 to generate an image demonstrating a movement trajectory of the target portion during one respiratory cycle. For example, the trajectory marking portion 223 may mark the target portion on each of a plurality of frames in the received ultrasound-model matched image. The trajectory marking portion 223 may generate an image demonstrating the movement trajectory of the target portion using the same method as the movement image generator 214 in generating the ultrasound-model matched image as described above. The image generated by the trajectory marking portion 223 may be an image having only the movement trajectory marked thereon except for the shape of the organ as illustrated in FIG. 10.

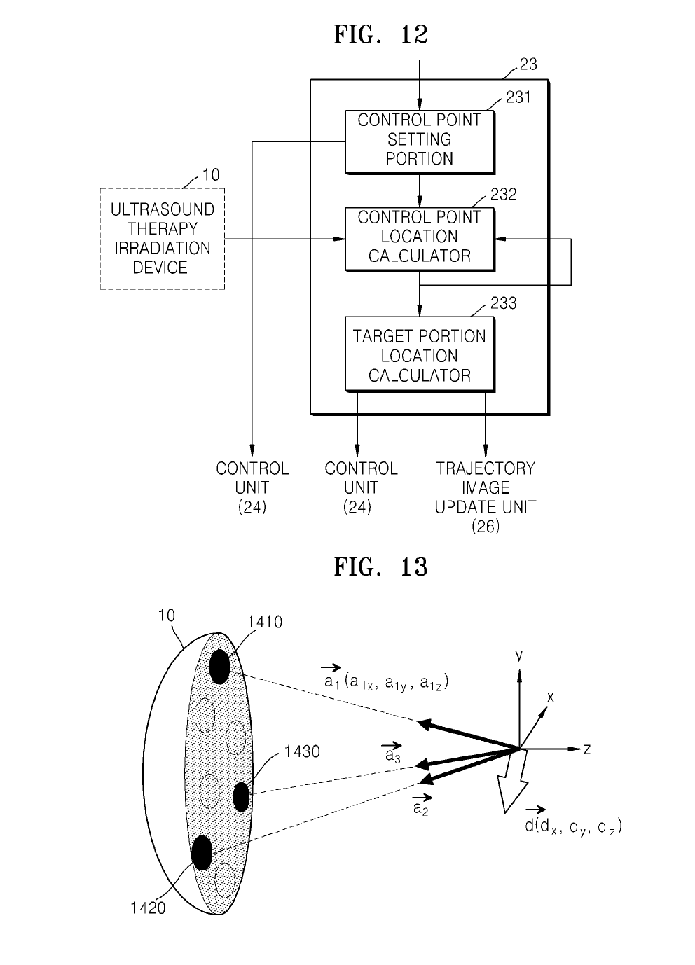

In an example, the image processing device 20 of FIG. 2 also includes a trajectory information acquiring unit 23 and a control unit 24. In this example, the therapeutic irradiation device 10 irradiates a control point located at a predetermined position in the patient's organ with a tracking ultrasound, and the trajectory information acquiring unit 23 uses a reflected ultrasonic wave to determine the location of at least one target portion. A method of determining the location of at least one target portion will now be described in more detail with reference to FIGS. 12 through 15.

FIG. 12 illustrates an example of the trajectory information acquiring unit 23. Referring to FIG. 12, the trajectory information acquiring unit 23 includes a control point setting portion 231, a control point location calculator 232, and a target portion location calculator 233.

In this examlple, the trajectory information acquiring unit 23 may first set a location of a control point, calculate a displacement of the control point during a respiratory cycle, use that displacement to calculate a displacement of a target portion, and transmit the displacement of the target portion to a control unit 24. The control unit 24 generates an ultrasound irradiation signal in response to the displacement of the target portion, and transmits the signal to the therapy irradiation device 10. The transmitted ultrasound controls irradiation by sub-apertures of the therapy irradiation device 10. That is, the sub-apertures irradiate the target portion with a therapeutic ultrasound in response to the received signal.

Referring to FIG. 12, the control point setting portion 231 sets a control point at a location near the target portion and sends information about the location of the control point to the control unit 24.The control point is a point near the target portion which is used as a reference point for tracking the movement of the target portion. Since the target portion is susceptible to changes, such as an increase in cell temperature or cell volume due to focusing of ultrasonic waves, a change in the transmission/reception of ultrasonic waves may result. Thus, the control point is set because it is difficult to accurately check whether the ultrasonic waves are constantly focused at a particular region. Since ultrasonic waves are focused on the target portion and not the control point, a cell located at the control point does not undergo a physical change.

In this example, some of the sub-apertures in the therapy irradiation device 10 transmit and receive tracking ultrasonic waves to and from the control point to continuously detect the location of the control point as it moves due to a patient's breathing. Also, the displacement of the target portion with respect to the control point is calculated so that the therapeutic ultrasonic waves are focused at the target portion. When a plurality of target portions are chosen, the process may be iteratively performed for each target portion.

In this example, the control unit 24 uses information about the control point received from the control point setting portion 231 to generate an ultrasound irradiation signal. The signal is transmitted to the ultrasound therapy irradiation device 10, and the sub-apertures of the therapy irradiation device 10 irradiate the location of the control point with a tracking ultrasound in response to the received signal.

A method of calculating a displacement at which a control point moves for one respiratory cycle in the control point location calculator 232 according to an embodiment of the present invention will now be described in detail with reference to FIG. 13.

FIG. 13 is a diagram illustrating an example of the calculation of a displacement of a control point using a triangulation algorithm. Referring to FIG. 13, three sub-apertures 1410-1430 in the therapy irradiation device 10 irradiate a control point with a tracking ultrasonic wave and receive a reflected wave from the control point. The control point is set at the origin of a coordinate axis. In this example, a displacement vector Δd of the control point is calculated using Equation (11):

$$Δ d = \frac{c}{2} {( {A}^{T} A )}^{- 1} Δ t \\ wherein A = ( \begin{matrix}{a}_{1 x} & {a}_{1 y} & {a}_{1 z} \\ {a}_{2 x} & {a}_{2 y} & {a}_{2 z} \\ ⋯ & ⋯ & ⋯ \\ {a}_{Nx} & {a}_{Ny} & {a}_{Nz}\end{matrix} ) , \\ Δ t = {( Δ {t}_{1} , Δ {t}_{2} , ⋯ , Δ {t}_{N} )}^{T} , and \\ Δ d = ( Δ {d}_{x} , Δ {d}_{y} , Δ {d}_{z} ) \qquad ( 11 )$$

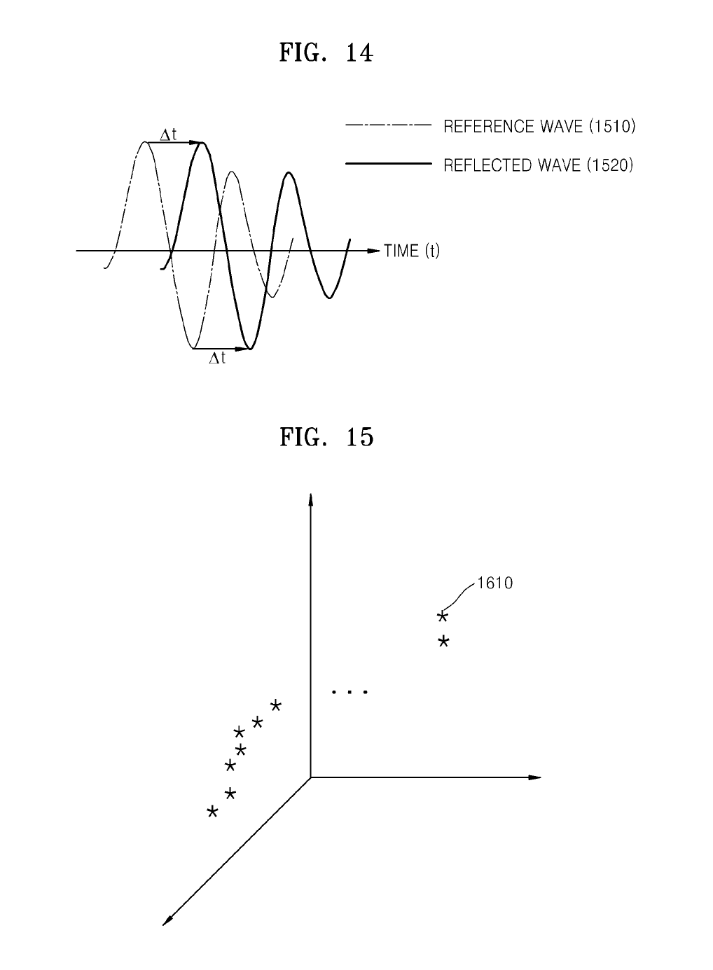

c and Δt denote in vitro ultrasound velocity and a matrix representation of the time shift tᵢ as measured by each sub-aperture, respectively. Referring to FIG. 14, the matrix Δt may be obtained by using a phase shift of a reflected wave 1520 with respect to a reference wave 1510. For example, the reference wave 1510 may be a reflected wave at a previous point in time. In this example, a point on the reflected wave 1510 and a corresponding point on the reference wave 1510 are compared, and the matrix Δt may be obtained by calculating a time difference between the two points.

The time shift tᵢ is defined by Equation (12):

$${t}_{i} = 2 \frac{{a}_{ix} dx + {a}_{iy} dy + {a}_{iz} dz}{c} \qquad ( 12 )$$

wherein aᵢ denotes a normalized vector indicating the direction from a control point towards an i-th sub-aperture and the direction of ultrasound for the i-th sub-aperture. The normalized vector aᵢ includes aᵢₓ, a_{iy}, and a_{iz}. In this example where N=3 such that i=1, 2, 3, a process of obtaining the displacement vector Δd is as follows. The control point calculator 232 calculates the time shifts t₁, t₂, and t₃ based on the information received from the three sub-apertures. The time shifts represent a difference between time measured when breathing stops and time measured during breathing. The normalized vector aᵢ pointing from the control point towards the i-th sub-aperture is determined according to the locations of the sub-apertures with respect to the control point as measured before movement. In other words, aᵢ is determined according to the relative location at an initial time point that is prior to the breathing cycle. c denotes in vitro ultrasound velocity. Thus, a simultaneous equation with three unknown variables dx, dy and dz may be obtained. By solving the simultaneous equation, Δd=(Δdₓ, Δd_{y}, Δd_{z}) may be obtained. The control point location calculator 232 calculates a current location of the control point by adding the initial location of the control point to the displacement vector Δd.

Referring to FIG. 12, information on the current location of the control point is transmitted to the target portion location calculator 233, which calculates a current location of the target portion using the current location of the control point. The movement trajectory of the target portion during a respiratory cycle may be obtained by determining the current location of the target portion at different predetermined time intervals throughout the respiratory cycle. Accordingly, combining the calculated locations at the different time intervals provides the movement trajectory of the target portion.

For example, referring to FIG. 15, locations 1610 of the target portion calculated at predetermined time intervals during one respiratory cycle are combined together providing the location of the target portion for the entire respiratory cycle. In this example, the target portion location calculator 233 calculates a current location of the target portion using the received current location of the control point. Since the control point setting portion 231 sets the control point at a location close to the target portion, the current location of the target portion may be obtained by using a distance between the target portion and the control point. That is, the difference in distance between the target portion and the control portion may be added to the current location of the control portion to determine the current location of the target portion. The current locations of the target portion may then be combined together to derive the location of the target portion throughout the entire respiratory cycle. The target portion location calculator 233 transmits the information about the current location of the target portion to the control unit 24 and the trajectory image update unit 26.

In this example, the control unit 24 receives the information from the target portion location calculator 233, and generates an ultrasound irradiation signal for sub-apertures to be irradiated with a therapeutic ultrasound in response to the current location of the target portion. The ultrasound irradiation signal is transmitted to the therapy irradiation device 10. The super-apertures within the therapy irradiation device 10 use the received signal to irradiate the target portion with the therapeutic ultrasound.

Situations where the movement information of the target portion, as obtained by the trajectory information acquiring unit 23, is not the same or is not expected to be the same as the movement trajectory of the target portion as determined by the trajectory image generation unit 22 will now be described in more detail with reference to FIGS. 2 and 16 through 18.

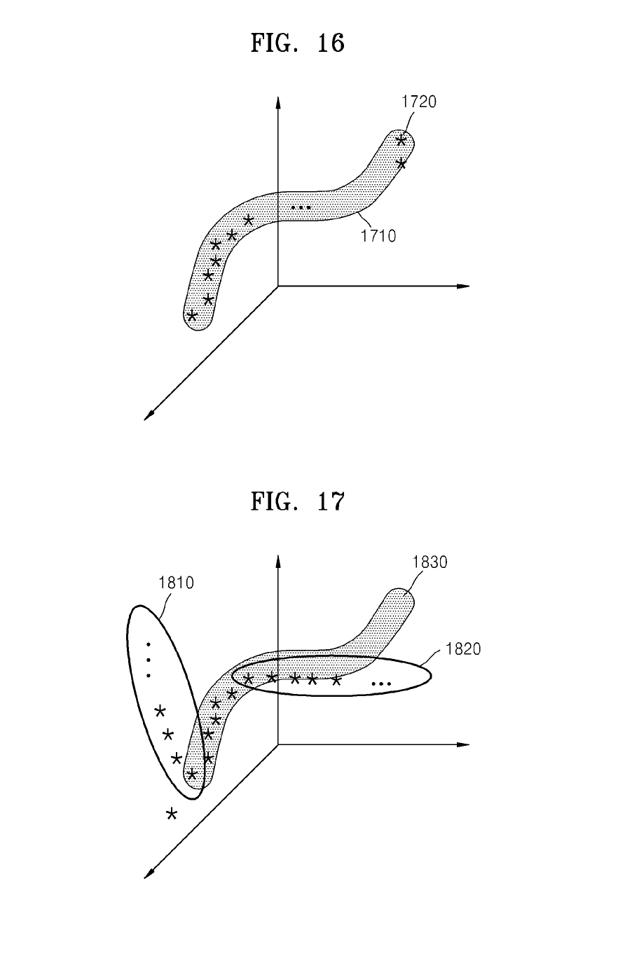

Referring to FIG. 2, the image processing device 20 also includes a trajectory image update unit 26. The trajectory image update unit 26 receives the image of the target portion from the image generation unit 22and received the information about the current location of the target portion from the trajectory information acquiring unit 23. Thereafter, the trajectory image update unit 26 indicates the movement information, i.e., the information about the current location of the target portion, on the image. For example, as illustrated in FIG. 16, the trajectory image update unit 26 transforms current location information 1720 with respect to a coordinate axis of the image showing the movement trajectory 1710 generated by the trajectory image generation unit 22. The trajectory image update unit 26 indicates the resulting information on the image so as to determine whether the movement information of the target portion is included in the movement trajectory 1710.

In an example, the image processing device 20 also includes a comparator unit 27. The comparator unit 27 determines whether the movement information 1720 of the target portion is included in the movement trajectory 1710 of the target portion during one respiratory cycle. That is, an error may occur when the information about the current location of the target portion is obtained. When the current location of the target portion deviates from the movement trajectory of the target portion due to the error, the ultrasound therapy irradiation device 10 may irradiate the wrong portion of the patient's body. Thus, when the current location of the target portion is outside the movement trajectory, the ultrasound therapy irradiation device 10 needs to stop irradiation of the therapeutic ultrasound.

In this example, the comparator unit 27 determines whether the information about the current location of the target portion deviates from or is expected to deviate from the movement trajectory. FIG. 17 illustrates an example of the current location information deviating from the movement trajectory. As shown in FIG. 17, the comparator unit 27 determines whether the current location of the target portion deviates from a movement trajectory 1830 of the target portion (as shown at 1810). Also, the comparator unit 27 determines whether the current location is expected to deviate from the movement trajectory 1830 (as shown at 1820). When such a deviation or expected deviation occurs, the comparator unit 27 generates a signal which notifies an operator of the HIFU system or a surgeon about the deviation, or can cease operation of the HIFU system. The generated signal is output to the image display device 30. For example, the comparator unit 27 may generate a signal for displaying a screen on the image display device which notifies about the deviation. Also, the comparator unit transmit a signal to the image display device 30 for emitting a warning sound to notify of the deviation. Alternatively, the comparator unit 27 may generate a signal for terminating the operation of the therapy irradiation device 10 and may transmit the signal to the control unit 24.

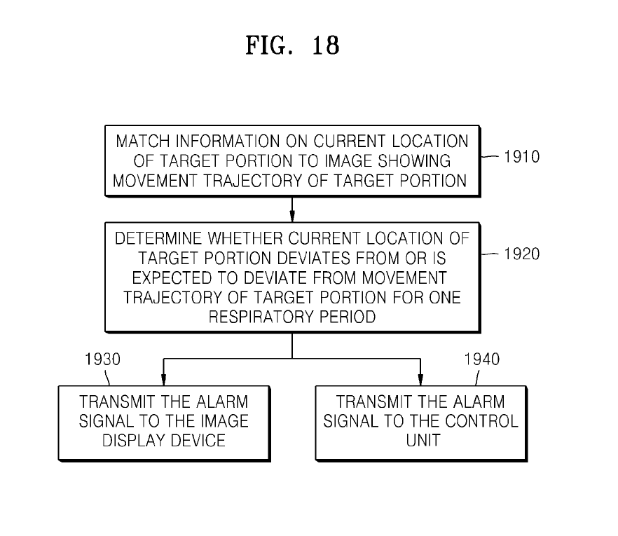

FIG. 18 is a flowchart illustrating an example of operations of the trajectory image update unit 26 and the comparator unit 27.

In operation 1910, the trajectory image update unit 26 matches information about a current location of a target portion to an image showing a movement trajectory of the target portion during one respiratory cycle by transforming the information about the current location with respect to a coordinate axis corresponding to a coordinate axis of the image.

In operation 1920, the comparator unit 27 determines whether the information about the current location of the target portion deviates from or is expected to deviate from the movement trajectory of the target portion during one respiratory cycle. When the information about the current location of the target portion deviates from or is expected to deviate from the movement trajectory, the comparator 27 generates an alarm signal and transmits the alarm signal to the image display device 30 (operation 1930) or to the control unit 24 (operation 1940). In When a control signal is sent to the control unit 24, the signal is used to terminate irradiation of a patient's body with the therapeutic ultrasound. When a control signal is sent to the image display device 30, the image display device 30 displays a screen that notifies of the deviation or emits a warning sound. In response to receiving the alarm signal, the control unit 24 may also generate a signal that ceases operation of the therapy irradiation device 10, and transmits the signal to the therapy irradiation device 10.

The therapy irradiation device 10, ultrasound irradiation device 60, image processing device 20, and image display device 30 described above may be implemented using one or more hardware components, or a combination of one or more hardware components and one or more software components. A hardware component may be, for example, a physical device that physically performs one or more operations, but is not limited thereto. Examples of hardware components include controllers, microphones, amplifiers, low-pass filters, high-pass filters, band-pass filters, analog-to-digital converters, digital-to-analog converters, and processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

Software or instructions for controlling a processing device, such as those described in FIGS. 5, 9, 11, and 12, to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of tracing a movement trajectory of a lesion in a patient's moving internal structure, comprising:
generating an image showing a movement trajectory of a target portion that is to be irradiated with a therapeutic wave;
irradiating the internal structure with a tracking wave;
determining a location of the target portion using a reflected wave; and
determining whether the location of the target portion is within the movement trajectory.

2. The method of claim 1, wherein:
the internal structure is a patient's organ;
the image shows a movement trajectory of the target portion during one respiratory cycle;
the image is generated based on a plurality of medical images of the patient's organ; and
the tracking wave is a tracking ultrasound wave and the therapeutic wave is a therapeutic ultrasound wave.

3. The method of claim 1, further comprising irradiating the target portion with the therapeutic wave according to a result of the determining whether the location of the target portion is within the movement trajectory.

4. The method of claim 1, further comprising generating an alarm signal representing cessation of irradiation of the therapeutic wave according to a result of the determining whether the location of the target portion is within the movement trajectory.

5. The method of claim 4, further comprising initiating either one or both of a visual indicator and an auditory indicator in response to the alarm signal.

6. The method of claim 1, further comprising:
predicting whether a next location of a plurality of locations of the target portion will be within the movement trajectory; and
irradiating the target portion with the therapeutic wave according to a result of the predicting.

7. The method of claim 2, wherein the generating of the image comprises:
selecting a landmark point from an average model of the organ; and
matching the average model of the organ to a medical image of the patient's organ by matching the landmark point to a corresponding location in the medical image.

8. The method of claim 2, wherein the image is a first image, and the generating of the first image comprises:
setting the target portion;
indicating the target portion on a second image that demonstrates a movement of the organ and contains unique anatomical information of the patient; and
generating the first image using the second image.

9. The method of claim 2, wherein the determining of the location of the target portion comprises:
setting a control point at a location in the patient's organ to be irradiated with the tracking ultrasound wave;
irradiating the control point with the tracking ultrasound wave and receiving the reflected wave;
calculating a location of the control point after the control point moves by using a phase shift of the received reflected wave; and
determining a location of the target portion after the target portion moves during one respiratory cycle using the calculated location of the control point.

10. A non-transitory computer-readable storage medium storing a program for controlling a computer to execute the method of claim 1.

11. A system for tracing a movement trajectory of a lesion in a patient's moving internal structure, the system comprising:
an irradiation device configured to irradiate the internal structure containing the lesion;
a trajectory image generation unit configured to generate an image showing a movement trajectory of a target portion that is a portion to be irradiated with a therapeutic wave;
a trajectory information acquiring unit configured to irradiate the internal structure with a tracking wave and determine a location of the target portion using a reflected wave; and
a comparator unit configured to determine whether the location of the target portion is within the movement trajectory.

12. The system of claim 11, wherein:
the internal structure is a patient's organ;
the image shows a movement trajectory of the target portion during one respiratory cycle;
the image is generated based on a plurality of medical images of the patient's organ; and
the tracking wave is a tracking ultrasound wave and the therapeutic wave is a therapeutic ultrasound wave.

13. The system of claim 11, wherein the comparator unit is further configured to generate an alarm signal representing cessation of irradiation of the therapeutic wave according to a result of the determining whether the location of the target portion is within the movement trajectory.

14. The system of claim 13, further comprising an image display device configured to initiate either one or both of a visual indicator and an auditory indicator in response to the alarm signal.

15. The system of claim 11, wherein the comparator unit is further configured to predict whether a next location of a plurality of locations of the target portion is within the movement trajectory and transmit a signal indicating irradiation of the target portion with the therapeutic wave to the ultrasound irradiation device according to a result of the predicting.

16. The system of claim 12, wherein the trajectory image generation unit is further configured to select a predetermined landmark point from an average model of the organ and match the average model of the organ to a medical image of the patient's organ by matching the landmark point to a corresponding location in the medical image.

17. The system of claim 12, wherein the image is a first image, and the trajectory image generation unit comprises:
a target portion setting unit configured to set the target portion; and
a trajectory marking portion configured to indicate the target portion on a second image that demonstrates a movement of the organ and contains unique anatomical information of the patient.

18. The system of claim 12, wherein the trajectory image generation unit comprises:
a control point setting portion configured to set a control point at a location in the patient's organ to be irradiated with the tracking ultrasound wave;
a control point location calculator configured to calculate a location after the control point moves using a phase shift of the reflected wave received in response to irradiating the control point with the tracking ultrasound wave; and
a target portion location calculator configured to determine a location of the target portion after the target portion moves during one respiratory cycle using the calculated location of the control point.

19. The system of claim 11, further comprising a trajectory image update unit configured to generate a mark indicating the location of the target portion on the image.

20. The method of claim 1, further comprising irradiating the internal structure with the therapeutic wave while performing the determining of whether the location of the target portion is within the movement trajectory.
